(19) United States
(12) Patent Application Publication
(10) Pub. No.: US 2004/0265344 A1
(43) Pub. Date: Dec. 30, 2004

(54) Aloe suppositories

(76) Inventors: Eyal Zolotariov, Tel Aviv (IL); Zvi Zolotariov, Tel Aviv (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/691,518

(22) Filed: Oct. 24, 2003

(30) Foreign Application Priority Data
Jun. 26, 2003 (IL) 156670

(51) Int. Cl. A61K 35/78; A61K 9/00

(52) U.S. Cl. 424/400; 424/744

(57) ABSTRACT

A suppository comprising aloe as a main active ingredient, intended for use as a laxative, and for treatment of hemorrhoids and bacterial infections of the anus, as an anti-inflammatory agent, anti-allergic agent and promoter of wound-healing. For the formulation of the present invention, the suppository preferably comprises aloe in an inert base, which may comprise any suitable inert pharmaceutical carrier. The base may optionally be any suitable inert base which is solid at room temperature. The aloe may optionally comprise the liquid or gel form or a dry extract of the juice, or any other form of aloe, all of which are collectively term “aloe extract”.

This application claims priority from Israel Patent Application No. 156670, filed Jun. 26, 2003, hereby incorporated by reference as if fully set forth herein.

### FIELD OF THE INVENTION

The present invention relates to suppositories, and more particularly to a suppository comprising aloe as the main active ingredient in an inert base, for treatment of constipation and hemorrhoids.

### BACKGROUND OF THE INVENTION

Constipation is the passage of small amounts of hard, dry bowel movements, usually fewer than three times a week, generally accompanied by pain which occurs when the colon absorbs too much water. This happens because the colon's muscle contractions are slow or sluggish, causing the stool to move through the colon too slowly. Constipation is the most common gastrointestinal complaint in the United States, resulting in about 2 million annual visits to the doctor. However, most people treat themselves without seeking medical help, as is evident from the $725 million Americans spent on non-prescription laxatives each year. Constipation may be due to a variety of causes, including insufficient dietary fiber intake, insufficient fluid intake, lack of exercise, or failure to respond promptly to an urge to defecate. Emotional and psychological problems can contribute to the problem. Constipation is also very common in pregnant women.

Some drugs and vitamin supplements can cause constipation: opiates such as morphine and codeine; aluminum salts in antacids; some dietary iron and calcium supplements; and certain antihistamines, diuretics, antidepressants, anti-psychotics and blood-pressure medications.

Persistent, chronic constipation may also be a symptom of more serious disorders, including irritable bowel syndrome, colorectal cancer, diabetes, Parkinson's disease, multiple sclerosis and depression.

Persistent constipation frequently leads to complications, such as hemorrhoids caused by straining to have a bowel movement or anal fissures caused when hard stool stretches the sphincter muscle. Hemorrhoids are specialized vascular areas lying subjacent to the anal mucosa. Symptomatic hemorrhoidal diseases are manifested by bleeding, thrombosis and/or prolapse of the hemorrhoidal tissues.

Constipation is commonly treated by use of laxatives. These may take the form of a glycerin suppository, which provides a mild irritant to help pass the stool.

Aloe plants are known natural laxatives. The pericylcic cells of the leaf produce a bitter yellow latex, which is a strong cathartic, containing various anthraquinones and their derivatives, the anthracenes. The major anthraquinones has been found to include barbaloin and aloin. The anthraquinones are water-soluble glycosides, easily separated from the water-insoluble resinous material of the leaves. The anthraquinones are colon-specific stimulant laxatives, which have a direct action on intestinal mucosa, increasing the rate of colonic motility, enhancing colonic transit, and inhibiting water and electrolyte secretion (Klinik et al., 1993; Gossel, 1991; Godding, 1988). The bioavailability of anthraquinone glycosides following oral administration has been shown to be poor (Reynolds 1991; Gilman et al 1990).

Aloe cathartic products may also have stool softening properties, and do not disrupt the usual pattern of defecation (Gilman et al, 1990; Godding, 1988).

Aloe-emodin-9-anthrone, a decomposition product of barbeloin, inhibited rat colonic mucosa sodium and potassium adenosine triphosphatase (ATP-ase) in vitro, and increased the paracellular permeability across the colonic mucosa of the rat. Multiple mechanisms are involved in the increased colonic movement, since loperamide prevented the increase of paracellular permeability, but did not completely inhibit the rise in residual fluid volume (Ishii et al, 1994; Ishii et al, 1994a; Ishii et al, 1990).

Treatment of constipation by aloe generally involves a single oral dose, administered at bedtime, although home remedies involving insertion of aloe vera leaves into the anus are known. Such home remedies result in use of an uncontrolled dosage, which is potentially dangerous. Oral doses of aloe may cause abdominal pains, gastro-intestinal irritation leading to pelvic congestion, and in large doses may result in nephritis, bloody diarrhea and hemorrhagic gastritis. Furthermore, excessive oral intake of aloe may be fatal. Aloe latex has been shown in vitro to be antibacterial against a number of gram-positive organisms. Since an abundance of bacteria are present in the environment of the rectum, a preparation having both laxative and antibacterial properties could be of considerable use in treatment of constipation and associated hemorrhoids.

The use of aloe in healing wounds, treating genital ulcers and eliminating hemorrhoids was recorded as early as 74 A.D. by the Greek physician Dioscorides (The Lawrence Review of Natural Products by Facts and Comparisons). Various constituents of aloe have also been shown to have anti-inflammatory and antibacterial effects, as well as to stimulate wound healing.

The inner parenchymal cells of the aloe plant leaf produce a slightly viscous, clear gel or mucilage. This gel is 96% water with various polysaccharides and sugars (galactose, xylose, arabinose and acetylated mannose), minerals, water-soluble and antioxidant vitamins (such as C and E), amino acids (essential and non-essential), enzymes (such as lipase, alkaline phosphatase, bradykinin-hydrolysing enzyme), lignin, beta-sitosterol, magnesium lactate, salicylic acid, succinic acid and various steroidal agents. When frozen, the gel becomes a red, gelatinous substance.

Aloe vera gel has traditionally been used in ointments and creams to assist the healing of wounds, burns, eczema and psoriasis.

The gel has been found to have antibiotic effects, which may be mediated by the sugar and polysaccharide components, via osmotic inhibition of bacterial growth.

Anti-inflammatory effects of the gel may be due to the salicylates, inactivation of bradykinin (via carboxypeptidases) and inhibition of histamine formation (Briggs, 1995; Natow, 1996). It appears that various non-specified components in the gel reduce the oxidation of arachidonic acid, thereby reducing prostaglandin synthesis and inflammation (Davis et al, 1987; Pennys, 1982).

Wound healing effects of the gel may involve inhibition of thromboxane and bradykinin. Allontoin, found in the gel, is known to stimulate epithelial cell development and proliferation.

Aloe vera gel has bactericidal activity against Bacillus subtilis, Citrobacter species, Enterobacter cloacae, Escherichia coli, Klebsiella pneumoniae, Mycobacterium tuberculosis, Pseudomonas aeruginosa, Serratia marcescens, Staphylococcus aureus, Streptococcus agalactiae, and Streptococcus faecalis. The antibacterial effect aids in the healing of anal wounds, such as hemhorroids, which may occur as a consequence of chronic constipation. Several prostanoid compounds have been found in aloe extracts. These prostanoids are produced from fatty acids by the enzyme cyclooxygenase. The major unsaturated fatty acid in the plant is gamma-linolenic acid, which can be converted to eicosatrienoic acid, the precursor to prostaglandins of the series, which are known to have beneficial effects in reducing inflammation and allergic reaction, and in increasing platelet aggregation and wound healing.

Extracts from the leaf gel and the rind have been shown to contain seven electrophoretically-identifiable superoxide dismutases. Other biologically active compounds found in aloe include a serine carboxypeptidase, salicylates, minerals, vitamins, sterols and amino acids.

Aloe is generally administered by either the oral or topical route, and is supplied in the form of capsules, gel, liquid, ointment or cream. Aloe has been approved by the FDA for use in the treatment of inflammation, and listed as a Category 1 agent (safe and effective) for skin protection. The American Herbal Products Association lists aloe as a stimulant laxative. Aloe barbadenis and Aloe capsenis are also approved by the German Commission E for use as a laxative.

### SUMMARY OF THE INVENTION

The background art does not teach or suggest a safe, modulated preparation of high stability containing an aloe component (extract of aloe), such as aloe juice and/or gel. The background art also does not teach or suggest such a preparation for relief of constipation and hemorrhoids, which also has anti-bacterial and anti-inflammatory actions, and which avoids the side-effects of the orally administered preparation. The background art teaches the use of aloe laxatives by oral administration which is associated with the disadvantages of low stability and various side effects, including abdominal pain and cramping.

The present invention overcomes these disadvantages of the prior art by providing a suppository comprising aloe as the main active ingredient in an inert base. In accordance with a preferred embodiment of the present invention, there is provided a pharmaceutical formulation in the form of a suppository, comprising an extract of aloe as the main active ingredient in a suitable inert pharmaceutical carrier.

A feature of the present invention is that the preparation is an effective laxative administered in the form of a suppository.

An advantage of the present invention is that the preparation exhibits high stability.

An additional advantage of the present invention is that the preparation also has anti-hemmorhoidal properties.

An additional advantage of the present invention is that administration of the preparation avoids the known side effects of the oral administration route.

An additional advantage of the present invention is that the preparation has antibacterial effects.

An additional advantage of the present invention is that the preparation has wound-healing actions.

### DETAILED DESCRIPTION

The present invention provides aloe as a main active ingredient in a suppository form, intended for use as a laxative, and for treatment of hemorrhoids and bacterial infections of the anus, as an anti-inflammatory agent, anti-allergic agent and promoter of wound-healing. For the formulation of the present invention, the suppository preferably comprises aloe in an inert base, which may comprise any suitable inert pharmaceutical carrier. The base may optionally be any suitable inert base which is solid at room temperature. The aloe may optionally comprise the liquid or gel form or a dry extract of the juice, or any other form of aloe, all of which are collectively term “aloe extract”.

The base may optionally and preferably comprise one or more emulsifiers such as PEG 40 stearate, PEG-100 stearate, glyceryl stearate, ceteareth 12, ceteareth 20, ceteareth 30, cetearyl alcohol, polyoxyethylene oleyl ether, or behentirmonium methosulphate. Optionally, the base may comprise one or more viscosity-adjusting agents such as microcrystalline wax, beeswax, paraffin or cetyl palmitate sodium stearate. Optionally and more preferably, the base comprises one or more preservatives such as methyl paraben or propyl paraben. Coloring material and/or fragrances may also optionally be added.

A preferred embodiment of the present invention comprises aloe in a glycerin base. Glycerin is preferred for use of the suppository of the present invention in treatment of constipation due to its osmotic properties. If glycerin is not included in the base, a higher dose aloe in the form of aloe vera or aloe ferox is preferably used. The dissolution properties of glycerin are well known, therefore no dissolution studies are included in herein.

Aloe as active ingredient may be used in the suppository of the present invention at doses higher than would be possible for oral administration. The recommended maximum daily dose is 600 mg. Each suppository may comprise 20-600 mg, therefore the maximum dose may be reached by administration of a single suppository containing 600 mg, or by use of two or three suppositories each containing smaller doses of active ingredient. Typical doses are 50-200 mg of active ingredient, administered up to thrice daily. Aloe may optionally comprise up to 30% of the total weight of the suppository. The suppository of the present invention may be produced by any standard method known to the art. Basically, this involves heating the base to the appropriate temperature, adding the active ingredients while mixing, transferring the composition to appropriate molds, and allowing it to cool.

The following are examples of compositions which can be employed in the formulation of the present invention. These examples are not intended to be limiting in any way:

### EXAMPLE 1

Active ingredients for this example are 2.92% water-based extract of aloe vera gel and 87.6% glycerin in a sodium stearate base formed by reaction of 1.82% sodium carbonate and 7.66% stearic acid at 100° C. This formulation provides a relatively rigid suppository having a high glycerin content.

### EXAMPLE 2

Active ingredients for this example include 16% water-extract of aloe vera gel in a 70% glycerin and 14% gelatin base.

### EXAMPLE 3

Active ingredients for this example include 70% glycerin, 3% water-based aloe vera gel extract, 13% water and 14% gelatin. The product is formed at temperatures of 40-60° C. The use of gelatin as carrier provides a more flexible suppository than that obtained with the composition of Example 1.

### EXAMPLE 4

Active ingredients for this example include a base of 50% polyethylene glycol (PEG) 6000 and 30% PEG 1540, with 20% water-based aloe vera gel extract.

### EXAMPLE 5

Active ingredients for this example include a hydrophobic hard fat base of between about 60 to about 97% hard fat Witepsol W45 and from 0% to about 23% hard fat Witepsol H15, with a dry extract of aloe vera of 0.8-23%.

In all of the above formulations, aloe comprises the main active ingredient.

These preparations have been shown to exhibit high stability for at least 6 months, and may be stored at temperatures of around 30÷C i.e. normal room temperature.

Tables 1 and 2 shows the results of stability monitoring studies performed on two batches of glycerin and aloe suppositories, of batch numbers 16602-G1 and 16602-G2 respectively, each of which were produced according to a formulation containing glycerin 2340 mg; aloe vera 78 mg; sodium carbonate 46 mg; and stearic acid 206 mg. The suppositories were provided in the form of light brown, transparent, molded suppositories, packaged in aluminum/aluminum foil (Alu/Alu), and stored at temperatures of 30° C. ±2° C., and at humidity of 60% RH ±5% RH in a climatic chamber.

Testing of microbial quality carried out at the beginning and end of the stability monitoring study revealed the suppositories to be free of Escherichia coli and essentially free of fungi and aerobic organisms.

TABLE 1
Stability monitoring study - batch no. 16602-G1

| | Initial | 1 month | 2 months | 3 months | 6 months |
|---|---|---|---|---|---|
| Water | 1.7% | 1.58% | 1.60% | 1.68% | 1.9% |
| determination | | | | | |
| Glycerin | 86% | 86.6% | 85.95 | 85.2 | 85.0 |
| assay | | | | | |

TABLE 2
Stability monitoring study - batch no. 16602-G2

| | Initial | 1 month | 2 months | 3 months | 6 months |
|---|---|---|---|---|---|
| Water | 2.23% | 1.94% | 2.09% | 1.95% | 2.3% |
| determination | | | | | |
| Glycerin | 86.2% | 86.2% | 85.5% | 85.4% | 86.8% |
| assay | | | | | |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A pharmaceutical formulation provided in the form of a suppository, comprising an extract of aloe as the main active ingredient in a suitable inert pharmaceutical carrier.

2. The pharmaceutical formulation according to claim 1 wherein said aloe is present as a water-based extract.

3. The pharmaceutical formulation according to claim 1 wherein said aloe is present as a dry extract.

4. The pharmaceutical formulation according to claim 2 further comprising glycerin.

5. The pharmaceutical formulation according to claim 1 wherein said inert carrier comprises sodium stearate.

6. The pharmaceutical formulation according to claim 5 wherein said sodium stearate is formed in place by reaction between sodium carbonate and stearic acid.

7. The pharmaceutical formulation according to claim 6 wherein said aloe extract is present in a concentration of about 3 percent.

8. The pharmaceutical formulation according to claim 7 wherein said glycerin is present in a concentration of about 87 percent.

9. The pharmaceutical formulation according to claim 6 wherein said sodium carbonate is present in a concentration of about 2 percent during at least one stage of said reaction.

10. The pharmaceutical formulation according to claim 6 wherein said stearic acid is present in a concentration of about 8 percent during at least one stage of said reaction.

11. The pharmaceutical formulation according to claim 1 wherein said carrier comprises gelatin.

12. The pharmaceutical formulation according to claim 11 wherein said aloe vera extract is present in a concentration of about 16 percent.

13. The pharmaceutical formulation according to claim 12 wherein said glycerin is present in a concentration of about 70 percent.

14. The pharmaceutical formulation according to claim 13 wherein said gelatin is present in a concentration of about 14 percent.

15. The pharmaceutical formulation according to claim 1 wherein said carrier comprises polyethylene glycol 6000.

16. The pharmaceutical formulation according to claim 15 wherein said carrier further comprises polyethylene glycol 1540.

17. The pharmaceutical formulation according to claim 15 wherein said aloe extract is present in a concentration of about 20 percent.

18. The pharmaceutical formulation according to claim 17 wherein said polyethylene glycol 6000 is present in a concentration of about 50 percent.

19. The pharmaceutical formulation according to claim 18 wherein said polyethylene glycol 1540 is present in a concentration of about 30 percent.

20. The pharmaceutical formulation according to claim 3 wherein said carrier comprises hard fat Witepsol W45.

21. The pharmaceutical formulation according to claim 20 wherein said carrier further comprises hard fat Witepsol H15.

22. The pharmaceutical formulation according to claim 20 wherein said aloe dry extract is present in a concentration of from about 0.8 to about 23%.

23. The pharmaceutical formulation according to claim 21 wherein said hard fat Witepsol W45 is present in a concentration of from about 15 to about 20%.

24. The pharmaceutical formulation according to claim 23 wherein said hard fat Witepsol H15 is present in a concentration of from about 0 to about 23%.

25. A suppository comprising as active ingredient an extract of aloe for use in treatment of constipation.

26. The suppository according to claim 25 wherein said extract of aloe comprises aloe juice.

27. The suppository according to claim 25 wherein said extract of aloe comprises aloe gel.

28. The suppository according to claim 25 wherein said extract of aloe comprises aloe gel and aloe juice.

29. The suppository of claim 27 for further use in treatment of hemorrhoids.

30. The suppository of claim 27 for further use as an antibacterial agent.

31. The suppository of claim 27 for further use in treatment of pain in the rectal area.

32. The suppository of claim 27 for further use in promotion of wound-healing in the rectal area.

33. The suppository of claim 27 for further use in treatment of inflammation in the rectal area.

34. The suppository of claim 27 for further use in treatment of allergic reaction in the rectal area.

35. A suppository comprising as active ingredient an extract of aloe gel.

36. The suppository of claim 35 for use in treatment of hemorrhoids.

37. The suppository of claim 35 for use as an antibacterial agent.

38. The suppository of claim 35 for use in treatment of pain in the rectal area.

39. The suppository of claim 35 for use in promotion of wound-healing in the rectal area.

40. The suppository of claim for use in treatment of inflammation in the rectal area.

41. The suppository of claim 35 for use in treatment of allergic reaction in the rectal area.
